(19) United States
(12) Patent Application Publication
(10) Pub. No.: US 2016/0137997 A1
(43) Pub. Date: May 19, 2016

(54) APPARATUS AND METHOD FOR DEHYDRATING BIOLOGICAL MATERIALS

(71) Applicant: Enwave Corporation, Vancouver (CA)

(72) Inventors: Timothy D. Durance, Vancouver (CA); Jun Fu, Port Coquitlam (CA)

(21) Appl. No.: 15/006,712

(22) Filed: Jan. 26, 2016

Related U.S. Application Data
(63) Continuation of application No. 13/265,277, filed on Oct. 19, 2011.
(60) Provisional application No. 61/173,566, filed on Apr. 28, 2009.

(51) Int. Cl. C12N 9/36; C12M 1/34; C12M 1/00

(52) U.S. Cl. CPC C12N 9/2462; C12M 21/14; C12M 41/40; C12M 31/00; C12M 47/14; C12Y 302/01017

(57) ABSTRACT

An apparatus and method for microwave vacuum-drying of temperature-sensitive biological materials on a continuous flow-through basis, in which the materials are frozen, ground to frozen particles, dehydrated to a powder, and the powder collected. The apparatus has a microwave generator and waveguide, a freezing chamber with a grinder, a rotatable dehydration chamber in or adjacent to the waveguide, and a powder collector to receive the powdered biological material. The apparatus operates under reduced pressure provided by a vacuum system coupled to the powder collector.

### FIELD OF THE INVENTION

The invention pertains to apparatuses and methods for microwave vacuum-drying of biological materials, in particular temperature-sensitive biological materials.

### BACKGROUND OF THE INVENTION

Many biologically-active materials, such as microbial cultures, proteins, enzymes, etc. are dehydrated for purposes of storage. Methods used in the prior art include freeze-drying and air-drying methods such as spray-drying. Dehydration generally lowers the viability of the materials. Freeze-drying allows higher viability levels than air-drying but it requires long processing times and is expensive.

It is also known in the art to dehydrate biological materials using microwave radiation in a vacuum chamber to remove water. When the materials are sensitive to damage at the elevated temperatures that can occur with microwaving, it is known to use a microwave freeze-drying process in which the material is frozen at low temperature in a vacuum chamber and the ice is sublimated by microwave radiation. Current systems are typically batch dehydrators, which limits efficiency. Also, current systems produce a dry “cake” from frozen solutions that must be subsequently milled to create a powder. Post-dehydration milling can produce excess heat and excess dust which can reduce biological activity and create handling difficulties, respectively.

### SUMMARY OF THE INVENTION

The invention provides an apparatus and method for dehydrating biological materials, employing freezing and microwaving. Examples of materials suitable for dehydration by means of the invention include bacterial suspensions, proteins, enzymes and other temperature-sensitive biological materials. Bacterial suspensions include many live-attenuated vaccines, dairy starter cultures, and other industrial starter cultures for fermentation processes. Proteins include milk proteins, egg proteins, soy proteins, and other plant and animal proteins, whether as isolates or in mixtures. Enzymes include proteases, trypsin, lysozyme, antibodies, immunoglobulins, amylases, cellulases, and other biological catalysts of industrial and medical importance. Other temperature-sensitive biological materials include deoxyribonucleic acid, ribonucleic acid, vegetable gums, antibiotics, and other complex organic molecules. Some plant extracts also benefit from freeze drying due to the presence of oxidation-susceptible components (e.g. ginseng extract) or unstable flavour components (e.g. coffee extract for soluble coffee, also known as instant coffee). The biological material, in an aqueous form such as a solution or suspension, is converted to frozen ice particles which are subjected to microwave vacuum-drying to form a powder, and the powder is conveyed to a collector.

The invention provides an apparatus for dehydrating an aqueous biological material having a microwave generator, a waveguide, and a freezing chamber for receiving the aqueous biological material and freezing it to form a frozen aqueous biological material. The apparatus includes means for feeding the aqueous biological material into the freezing chamber, means for forming a particulate frozen aqueous biological material from the frozen aqueous biological material, a dehydration chamber in fluid communication with the freezing chamber, and a powder collector in fluid communication with the dehydration chamber. A vacuum system is operatively connected to the powder collector for applying a vacuum to the freezing chamber, the dehydration chamber and the powder collector.

The invention further provides an apparatus for dehydrating an aqueous biological material having a microwave generator, a waveguide, and a freezing chamber for receiving and freezing the aqueous biological material. The apparatus includes means for feeding the aqueous biological material into the freezing chamber, a grinder in the freezing chamber, a rotatable dehydration chamber in fluid communication with the freezing chamber, and a powder collector in fluid communication with the dehydration chamber. Free-moving mill balls may be provided within the freezing chamber and/or the dehydration chamber. A vacuum system is operatively connected to the powder collector for applying a vacuum to the freezing chamber, the dehydration chamber and the powder collector.

The invention further provides a method for dehydrating an aqueous biological material. The aqueous biological material is fed into a freezing chamber. The aqueous biological material is caused to freeze to a frozen material under reduced pressure in the freezing chamber. The frozen material is ground to a particulate frozen material. The particulate frozen material is conveyed into a rotatable dehydration chamber. The biological material may be further reduced in size by the grinding action of free-moving balls within the freezing chamber and/or the dehydration chamber. The dehydration chamber is rotated or oscillated and the particulate frozen material is microwaved under reduced pressure in the dehydration chamber to sublimate water from the material, leaving the biological material in powder form. The powder is conveyed from the dehydration chamber to a powder collector.

The invention further provides a method for dehydrating an aqueous biological material. The aqueous biological material is fed into a freezing chamber. A particulate frozen material is formed from the aqueous biological material. The particulate frozen material is conveyed into a dehydration chamber and is microwaved under reduced pressure in the dehydration chamber to sublimate water from the material, leaving the biological material in powder form. The dried powder is selected and conveyed from the dehydration chamber to a powder collector. The dehydration chamber may be rotated during the microwaving.

These and other features of the invention will be apparent from the following description and drawings of the preferred embodiment.

### BRIEF DESCRIPTION OF DRAWINGS

FIG. 1 is an isometric view of an apparatus according to one embodiment of the invention.

FIG. 2 is a cross-sectional view thereof on the line 2-2 of FIG. 1.

FIG. 3 is a schematic, cross-sectional view thereof on the line 3-3 of FIG. 1.

FIG. 4 is a sectional view of the freezing chamber.

FIGS. 5 and 6 are isometric, partly cutaway views of an apparatus according to a second embodiment of the invention.

### DESCRIPTION OF THE PREFERRED EMBODIMENTS

### First Embodiment of the Dehydrating Apparatus

In the following description and the drawings, like and corresponding elements are identified by the same reference numerals.

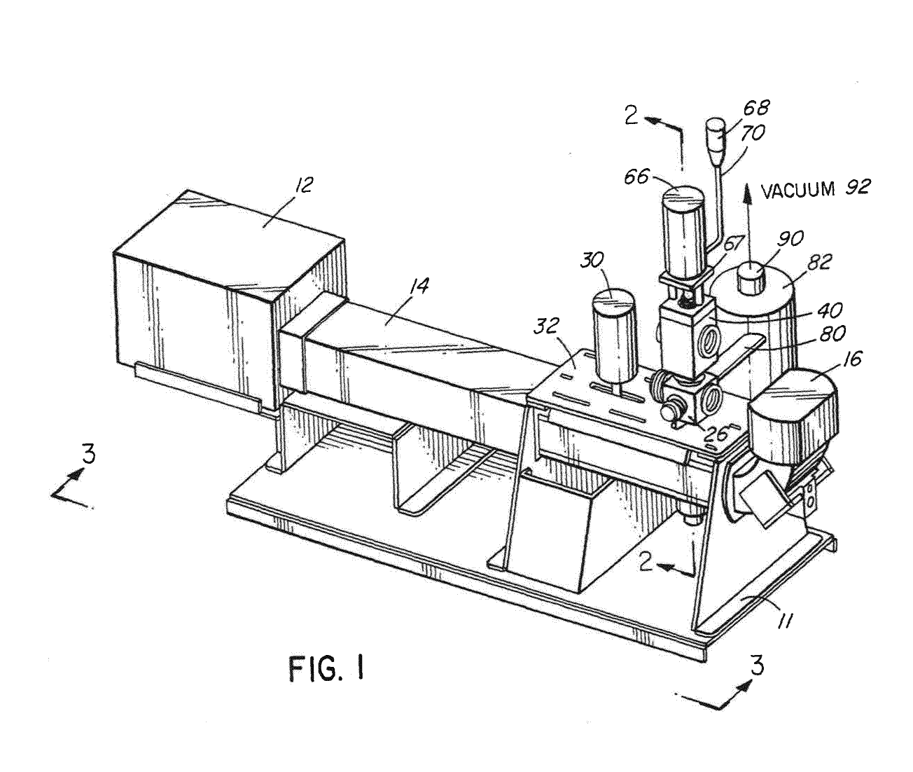

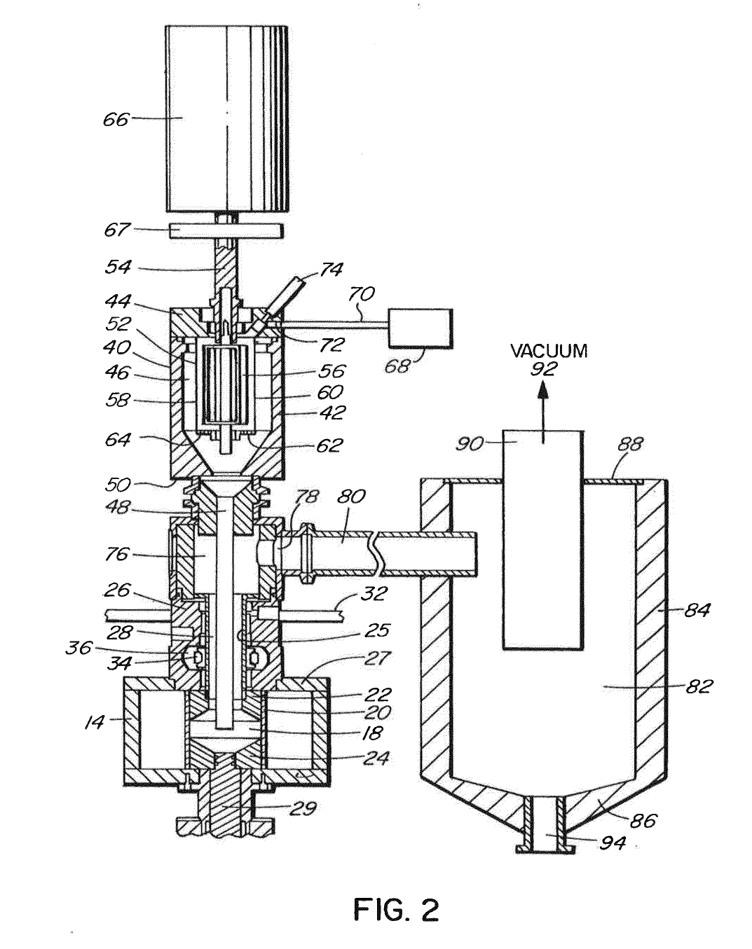

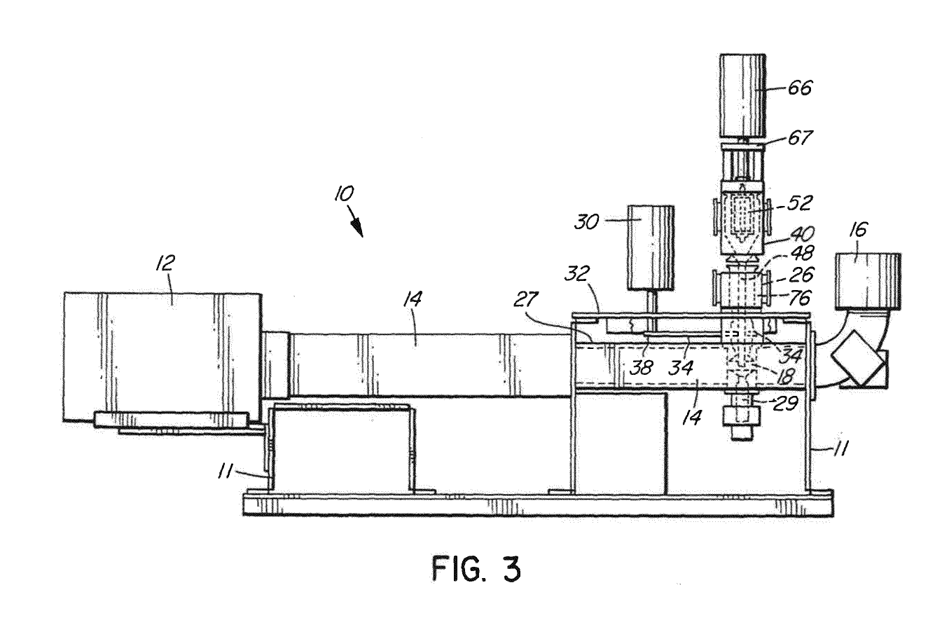

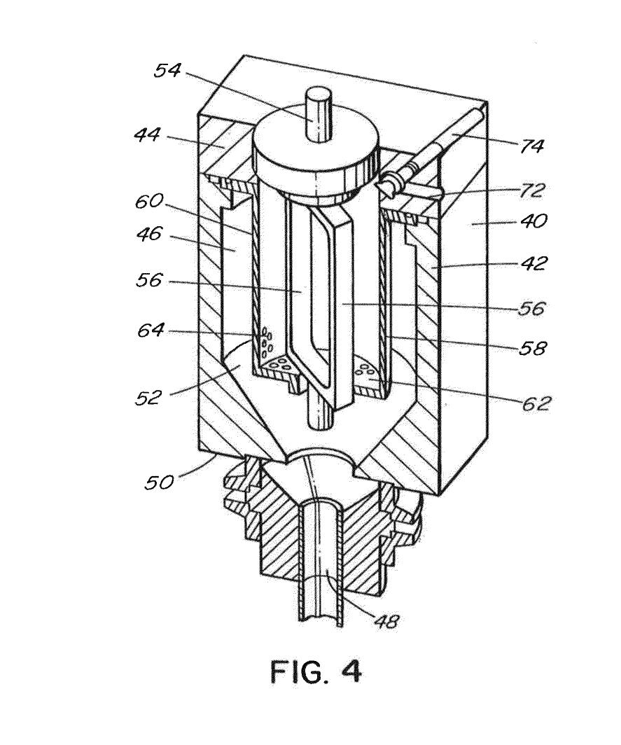

Referring to FIGS. 1 to 4, the dehydration apparatus 10 has a microwave generator 12, a tubular waveguide 14 and a water load 16, supported on a stand 11 and arranged so that microwave radiation from the generator travels through the waveguide and is absorbed by the water load.

A rotatable dehydration chamber 18 is located in the waveguide 14. It has a microwave-transparent body comprising a cylindrical side wall 20, an upper body portion 22 and a lower body portion 24. A mounting block 26 is fitted into the upper wall 27 of the waveguide. The dehydration chamber is rotatably connected to the mounting block 26 with a rotatable sleeve 25 arranged vertically in the mounting block and attached to the dehydration chamber. A motor 30 is mounted on a support plate 32 above the waveguide upper wall 27. A drivebelt 34 extends through a slot 36 in the mounting block from the pulley 38 of the motor 30 to engage the sleeve 25. The sleeve 25 forms an annular channel 28 within the mounting block 26 for the transport of powder from the dehydration chamber. A rotatable shaft 29 with bearings connected to the lower body portion 24 of the dehydration chamber stabilizes the rotation of the dehydration chamber. Optionally, the apparatus includes means for periodically reversing the direction of rotation of the dehydration chamber. This permits the chamber to oscillate.

A grinder housing 40 is mounted on top of the mounting block 26. It has a side wall 42, a removable upper wall 44 and defines within it a freezing chamber 46. An ice conduit 48 is attached to the bottom side 50 of the grinder housing, extending from the freezing chamber 46 through the mounting block 26 and sleeve 25 into the dehydration chamber 18.

A grinder 52 is located in the freezing chamber 46. It comprises a shaft 54 with two spaced blades 56 mounted thereon within a perforated grinder body 58 having a cylindrical side wall 60 and bottom wall 62, both of which have a plurality of perforations 64. A grinder motor 66 is mounted on a support plate 67, which is supported by legs 69 on the grinder housing upper wall 44. The grinder shaft 54 extends through a bore in the grinder housing upper wall and is connected to the grinder motor for rotation thereby.

Optionally, free-moving mill balls (not shown) may be provided within the freezing chamber, the dehydration chamber or both. In the dehydration chamber, the mill balls provide an action similar to that of a ball mill, assisting in forming fine powders. The action of the balls also keeps residues from building up in the dehydration chamber, thus eliminating potential fouling. In the freezing chamber, within the grinder body 54, free-moving mill balls assist in size-reduction of the frozen material and also prevent fouling. The mill balls may be made of ceramic, quartz or other hard material with a sufficiently low dielectric loss factor so as not to heat in the microwave field.

A feedstock supply vessel 68 for the aqueous biological material to be processed is connected by a conduit 70 to an inlet port 72 in the upper wall 44 of the grinder housing, whereby the feedstock is fed into the freezing chamber 46. A feedstock flow controller 74 is connected to the inlet 72 for regulation of the rate of flow of the feedstock.

The mounting block 26 defines a chamber 76 which is open from its lower side to the annular channel 28. The ice conduit 48 extends through this chamber 76 and through the sleeve 25. The chamber 76 is open on one side through a powder outlet port 78. A powder outlet conduit 80 connects the outlet port 78 to a powder collector 82. This collector comprises a closed vessel having a cylindrical side wall 84, a bottom wall 86 and a lid 88. Powder is removed from the powder collector by gravity, that is by falling through the powder collector outlet 94 into a reservoir chamber or chambers (not shown). Powder may be directed to alternate reservoirs by a selector valve to allow periodic emptying of the reservoirs. The powder outlet conduit 80 extends into the powder collector through its side wall. A vacuum inlet tube 90 extends through the lid 88 into the powder collector and is connected to a vacuum pump 92, or other vacuum source, and a water condenser (not shown).

The freezing chamber 46, dehydration chamber 18, powder collector 82 and the passageways that connect them form a closed system, and accordingly the application of vacuum to the vacuum inlet tube 90 creates a low pressure state throughout the system. Typical operating pressures are in the range of 0.1 to 1.0 mm of mercury absolute pressure.

The apparatus 10 also includes a controller (not shown) such as a PLC (programmable logic computer) to operate the system, including controlling the inflow of feedstock, the microwave output, the vacuum system, and the rotation of the grinder and the dehydration chamber.

The dehydrating apparatus 10 operates according to the following method. First, the aqueous biological material feedstock is prepared and loaded in the feedstock supply vessel 68. For example, the feedstock solution may be pre-concentrated by vacuum evaporation to a viscous liquid. Bacterial cultures or other liquid suspensions may be propagated in a fermentation vessel, then concentrated by centrifugation to approximately 20% solids. The vacuum pump 92, the microwave generator 12, the grinder motor 66 and the dehydration chamber motor 30 are actuated. The aqueous biological material is fed into the freezing chamber 46. The material immediately freezes to ice under the reduced pressure. The grinder grinds the frozen material to ice particles, which pass through the perforations 64 in the grinder body 58 and descend through the ice conduit 48 into the spinning dehydration chamber 18. The microwave radiation passing through the waveguide sublimates the ice to water vapor, leaving the biological material in the chamber 18 as a dry powder. Optionally, free-moving balls in the freezing chamber and/or the dehydration chamber assist in forming fine powder. As water vapor from the sublimated ice is drawn toward the vacuum inlet tube 90, the powder is drawn with it through the annular powder channel 28, the chamber 76 and the powder outlet conduit 80, and is deposited into the powder collector 82. The water vapor exits the powder collector through the vacuum inlet tube 90. The vacuum system delivers the water vapor to the condenser to be condensed and frozen to solid water.

The system operates on a continuous throughput basis, with collected powder being removed periodically from the powder collector.

### Second Embodiment of the Dehydrating Apparatus

In the dehydration apparatus 10 described above, the grinder shaft 54 and the dehydration chamber 18 are rotatable about an axis that is substantially vertical. The invention includes dehydrating apparatuses in which this axis of rotation is not vertical. For example, it may be horizontal or have a slope.

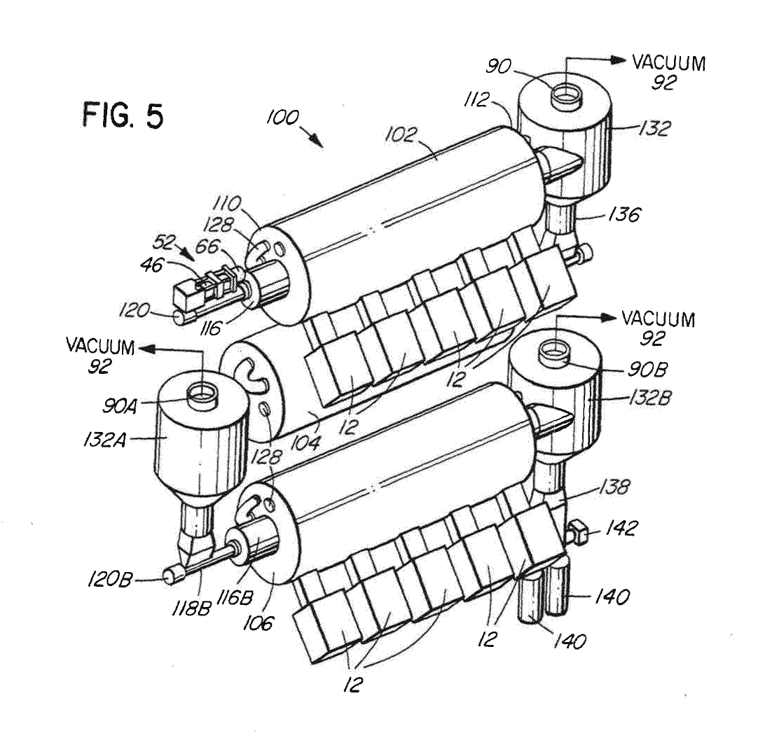

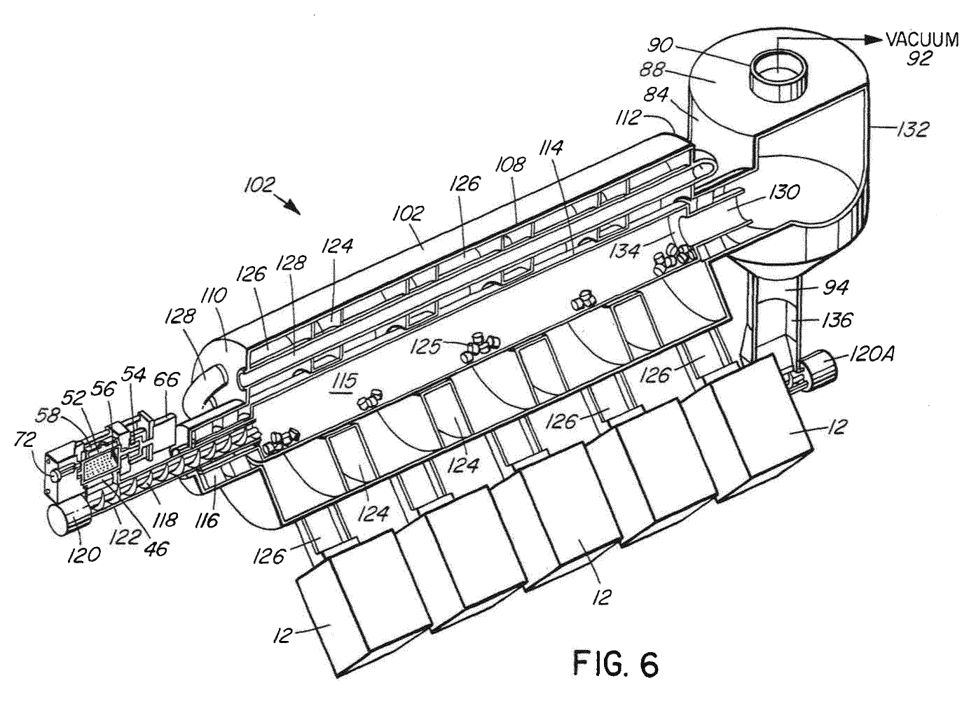

FIGS. 5 and 6 illustrate a dehydration apparatus 100 in which this axis of rotation is substantially horizontal. The dehydration apparatus 100 comprises three dehydration units 102, 104, 106 arranged in series. The first dehydration unit 102 has a housing 108 with an input end 110 and an output end 112. A microwave-transparent tube 114 extends longitudinally through the unit and is rotatable about its longitudinal axis by a motor 116. The tube 114 defines a dehydration chamber 115.

A freezing chamber 46 with a grinder 52 for grinding ice is provided at the input end 110 of the tube 114. The grinder has grinder blades 56 rotatable within a grinder body 58 by a grinder motor 66.

The dehydration apparatus 100 has a feedstock supply system (not shown) which is the same as that described above for the dehydration apparatus 10, namely a feedstock supply vessel, feedstock flow controller and an inlet conduit, for delivering aqueous biological material to an inlet port 72 of the freezing chamber 46.

An auger 118, rotatable by a motor 120 in an auger tube 122 is positioned under the freezing chamber 46 to receive ice particles from the grinder and feed them into the input end of the dehydration chamber 115. Optionally, the freezing chamber 46 or the dehydration chamber 115, or both, may be provided with free-moving mill balls 125.

The dehydration unit 102 includes a set of microwave generators 12, five in the illustrated embodiment, connected to waveguides 126 which extend circumferentially around the tube 114 between the housing 108 and the tube 114. The waveguides 126 are separated by circumferential spaces 124. Water circulation tubes 128 extend longitudinally through the space between the housing 108 and the tube 114, passing through the waveguides 126. A pump (not shown) pumps water through the water tubes 128. The water acts as a water load for absorbing energy and carrying away heat.

The dehydration chamber 115 is open at the outlet end 112 of the dehydration unit 102, with an outlet conduit portion 130 of the tube extending into a powder collector 132. The conduit portion 130 has a lip 134 at its inward end which prevents the mill balls from entering the powder collector. Alternatively, a screen can be provided for this purpose at the inward end of the conduit portion 130. A vacuum inlet tube 90 extends through the lid 88 of the powder collector 132 and is connected to a vacuum source and water condenser (not shown). A powder outlet conduit 136 extends from the bottom side of the powder collector 132. At its lower end, the conduit 136 is open to the auger 118A of the second dehydration unit 104.

The second dehydration unit 104 and the third dehydration unit 106 have the same structure as the first unit 102. They feed powder into powder collectors 132A and 132B respectively, which have vacuum inlet tubes 90A and 90B respectively, connected to the vacuum source and water condenser. Powder produced by the first unit 102 is fed into the second unit 104 by the auger 118A, rotated by a motor 120A. The powder that exits the second unit 104 enters the second powder collector 132A and is delivered by an auger 118B to the third dehydration unit 106. The powder that exits the third unit 106 enters the third powder collector 132B. A chute extends from the bottom side of the powder collector 132B to the powder receptacles 140. A selector valve 142 between the chute 138 and the receptacles allows for the periodic removal and emptying of the receptacles 140.

The dehydrating apparatus 100 has been described and illustrated as comprising three dehydration units in series. However, it can comprise any selected number, for example one, two, or four or more. This is a matter of design choice, dependent upon the desired dehydration capacity, final moisture content, type of biological material and particle size. For example, larger particles may require longer microwave exposure at a lower power to achieve the same final moisture content, while hydroscopic compounds such as simple sugars may require longer microwave exposure than less hydroscopic compounds such as large molecular weight polysacchardes.

The dehydrating apparatus 100 operates according to the following method. The vacuum pump, water pump, microwave generators 12, grinder motor 66, three auger motors 120, 120A, 120B, and the dehydration chamber motors 116, 116A, 116B are actuated. The dehydration chamber motors 116, 116A, 116B may be operated at different rotation speeds, and the respective sets of microwave generators 12 of each of the units 102, 104, 106 may be operated at different power levels. For example, the microwave power level may be highest in the first unit 102, lowest in the third unit 106 and intermediate in the second unit 104. The dehydration chamber rotation speed may be highest in the first unit 102, lowest in the third unit 106 and intermediate in the second unit 104. The settings are selected to optimize the drying of the powder, the object being to obtain fully dried powder in the receptacles 140 after processing in all three units.

The aqueous biological material is fed into the freezing chamber 46. The material immediately freezes to ice under the reduced pressure. The grinder grinds the frozen material to ice particles, which pass through the perforations in the grinder body 58 and fall into the auger tube 122. The auger 118 moves the particles into the rotating dehydration chamber 115. Microwave radiation passing through the waveguides 126 passes through the microwave-transparent tube 114 and sublimates the ice to water vapor, leaving partially dried, powdered biological material in the chamber. Optionally, there are free-moving mill balls in the freezing chamber and/or the dehydration chamber which assist in forming fine powder.

As water vapor is drawn toward the vacuum inlet tube 90, the powder is drawn with it through the chamber 115, outlet conduit 130 and into the powder collector 132. To assist the movement of powder through the chamber 115, vanes may optionally be provided on the inner wall of the tube 114, or the dehydration unit may optionally be sloped downward from the input end to the output end, whereby movement of the powder toward the outlet end is assisted by gravity.

From the powder collector 132, the powder descends through the conduit 136 to the auger 118A of the second unit 104. The drying process continues in the same manner in the second and third units 104, 106, delivering fully dried powder to the powder receptacles 140. When one receptacle 140 is full, the selector valve 142 directs powder to an empty receptacle, and the filled receptacle is removed. The system is operated on a continuous throughput basis.

### EXAMPLE

A dehydration apparatus in the form of the apparatus 10 described above has a microwave generator with a power output of 500 watts. The vacuum system evacuated the apparatus to an absolute pressure of 0.20 mm of mercury. The dehydration chamber was rotated at 300 rpm and the grinder at 100 rpm. A 20% solution by weight of chicken lysozyme in water was applied as the feedstock at a rate of 0.4 mL per minute. The apparatus was operated according to the method described above, producing outlet powder with a moisture content of 4.53%. Lysozyme activity retention was almost entirely retained in the dried product.

Although the invention has been described in terms of particular embodiments, it is not intended that the invention be limited to these embodiments. Various modifications within the scope of the invention will be apparent to those skilled in the art. For example, instead of spinning the dehydration chamber, an impeller or other form of agitator may be provided in the chamber to induce the flow of dehydrated powder therefrom. Further, instead of forming ice particles by means of grinding, a spraying or atomizing system can be employed to form droplets of the feedstock which freeze to ice particles and do not require grinding to be in a suitable form to flow into the dehydration chamber and be microwaved.

### LIST OF REFERENCE NUMERALS IN THE DRAWINGS

10 dehydration apparatus
11 stand
12 microwave generator
14 waveguide
16 water load
18 dehydration chamber
20 side wall of dehydration chamber
22 upper body portion of dehydration chamber
24 lower body portion of dehydration chamber
25 rotatable sleeve
26 mounting block
27 upper wall of waveguide
28 annular powder channel
29 shaft with bearings
30 motor for dehydration chamber
32 support plate
34 drivebelt
36 pulley slot in mounting block
38 motor pulley
40 grinder housing
42 side wall of grinder housing
44 upper wall of grinder housing
46 freezing chamber
48 ice conduit
50 bottom side of grinder housing
52 grinder
54 grinder shaft
56 grinder blades
58 grinder body
60 side wall of grinder body
62 bottom wall of grinder body
64 perforations in grinder body
66 grinder motor
67 support plate
68 feedstock supply vessel
69 support legs
70 feedstock conduit
72 feedstock inlet port
74 feedstock flow controller
76 chamber in mounting block
78 outlet port in mounting block
80 powder outlet conduit
82 powder collector
84 powder collector side wall
86 powder collector bottom wall
88 powder collector lid
90, 90A, 90B vacuum inlet tubes
92 vacuum pump
94 powder collector outlet
100 dehydration apparatus
102 first dehydration unit
104 second dehydration unit
106 third dehydration unit
108 housing of dehydration unit
110 input end of dehydration unit
112 output end of dehydration unit
114 rotatable tube
115 dehydration chamber
116, 116A, 116B motors for dehydration chambers
118, 118A, 118B augers
120, 120A, 120B auger motors
122 auger tube
124 space between waveguides
125 mill balls
126 waveguides
128 water circulation tubes
130 outlet conduit of dehydration chamber
132, 132A, 132B powder collectors
134 lip of outlet conduit
136 powder outlet conduit
138 powder chute
140 powder receptacles
142 selector valve

1. An apparatus for continuous throughput dehydration of an aqueous biological material, comprising:
(a) a microwave generator;
(b) a waveguide to direct microwave radiation from the generator;
(c) a freezing chamber for receiving the aqueous biological material and freezing it to form a frozen aqueous biological material;
(d) means for feeding the aqueous biological material into the freezing chamber;
(e) means for forming a particulate frozen aqueous biological material from the frozen aqueous biological material;
(f) a rotatable dehydration chamber in fluid communication with the freezing chamber, the dehydration chamber being positioned in the waveguide to receive microwave radiation produced by the generator, the dehydration chamber having a wall that is transparent to microwave radiation;
(g) means for rotating the dehydration chamber;
(h) a powder collector in fluid communication with the dehydration chamber;
(i) a vacuum system; and
(j) means for operatively connecting the vacuum system to the powder collector for applying a vacuum to the freezing chamber, the dehydration chamber and the powder collector whereby the vacuum conveys dehydrated biological material from the dehydration chamber to the powder collector on a continuous throughput basis.

2. An apparatus according to claim 1, further comprising an agitator in the dehydration chamber.

3. An apparatus according to claim 1, further comprising free-moving mill balls in the dehydration chamber.

4. An apparatus according to claim 1, wherein the means for forming a particulate frozen aqueous biological material comprises a grinder.

5. An apparatus according to claim 1, wherein the means for forming a particulate frozen aqueous biological material comprises a sprayer.

6. An apparatus according to claim 5, wherein the grinder is positioned within the freezing chamber.

7. An apparatus according to claim 1, further comprising free-moving mill balls in the freezing chamber.

8. An apparatus according to claim 1, further comprising the vacuum system.

9. An apparatus according to claim 1, further comprising a second dehydration chamber having an inlet end operatively connected to the powder collector, and having a second powder collector at an outlet end of the second dehydration chamber.

10. An apparatus according to claim 10, wherein the dehydration chambers comprise tubes oriented substantially horizontally.

11. An apparatus according to claim 10, further comprising means for operatively connecting the vacuum system to the second powder collector.

12. An apparatus according to claim 10, further comprising a third dehydration chamber having an inlet end operatively connected to the second powder collector, and having a third powder collector at an outlet end of the third dehydration chamber.

13. An apparatus according to claim 1, wherein the aqueous biological material comprises a bacterial suspension, a protein, an enzyme, deoxyribonucleic acid, ribonucleic acid, a vegetable gum, or an antibiotic.

14. A method for continuous throughput dehydration of an aqueous biological material, comprising the steps of:
(a) providing an apparatus having a freezing chamber, a rotatable dehydration chamber and a powder collector in fluid communication;
(b) applying a vacuum to the powder collector whereby pressure is reduced in the freezing chamber, the dehydration chamber and the powder collector;
(c) feeding the aqueous biological material into the freezing chamber;
(d) forming a particulate frozen material from the aqueous biological material;
(e) conveying the particulate frozen material from the freezing chamber into the dehydration chamber;
(f) microwaving the particulate frozen material under reduced pressure in the dehydration chamber, while rotating the dehydration chamber, to sublimate water from the aqueous biological material, to produce a powdered biological material; and
(g) conveying the powdered biological material from the dehydration chamber to the powder collector on a continuous throughput basis by means of the vacuum applied to the powder collector.

15. A method according to claim 15, wherein the step of forming the particulate frozen material comprises freezing the aqueous biological material and grinding the frozen material.

16. A method according to claim 15, further comprising the step of agitating the powder in the dehydration chamber.

17. A method according to claim 17, wherein the step of agitating is done by means of mill balls in the dehydration chamber.

18. A method according to claim 15, further comprising the step of periodically reversing the direction of rotation of the dehydration chamber.
